(19) United States
(12) Patent Application Publication
(10) Pub. No.: US 2005/0232915 A1
(43) Pub. Date: Oct. 20, 2005

(54) Matrix metalloproteinase inhibitors in combination with hypothermia and/or radiotherapy for the treatment of cancer

(76) Inventors: Motowo Nakajima, Tokyo (JP); Kazuhiro Mizumoto, Fukuoka-ken (JP); Li-Wu Qian, San Antonio, TX (US); Hiroyuki Honda, Aichi-ken (JP); Naoki Kato, Gifu-ken (JP); Takeshi Kobayashi, Aichi-ken (JP)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/506,936

(22) PCT Filed: Mar. 7, 2003
(86) PCT No.: PCT/EP03/02365

§ 371 Date: Jun. 6, 2005

(30) Foreign Application Priority Data
Mar. 8, 2002 (GB) 0205537.4
Dec. 12, 2002 (GB) 0229054.2

(51) Int. Cl. A61K 38/46; A61K 31/44; A61K 31/19

(52) U.S. Cl. 424/94.65; 514/19; 514/357; 514/575

(57) ABSTRACT

The invention provides a method of treating cancer in a subject in need of such treatment which comprises: radio-therapy, or cytotoxic therapy in combination with heat shock, and further comprises administering to the subject an effective amount of a matrix metalloproteinase.

This invention relates to organic compounds, in particular to pharmaceutical compositions for use in combination with cytotoxic therapy and heat shock for the treatment of tumors.

Carcinoma is by far the most common type of cancer; it accounts for about 80% of all cases of cancer. The severity of a carcinoma can vary widely with pancreatic cancer being one of the most aggressive and lethal neoplasms with an extremely low 5-year survival rate; Landis, S. et al (CA Cancer J. Clin., 49: 8-31, 1999) and Niederhuber, J. E. et al (Cancer, 76:1671-1677, 1995). Because most patients with pancreatic cancer miss the opportunity for complete surgical resection at the time of diagnosis, radiotherapy remains as a major component of treatment modalities for controlling tumor progression. Malignant progression of pancreatic cancer depends not only on rapid proliferation of tumor cells but also on other biological behaviours including motility, invasiveness, and metastatic potential. More generally radiotherapy remains a major therapeutic option for patients with various other types of advanced cancer. Radiotherapy besides having the desired effect also has an effect on malignant biological behaviours for example it has now been found that while it significantly inhibits cell proliferation and migration irradiation may enhance the invasive potential in pancreatic cancer cells.

Current treatments for cancer are effective to some extent but all have some undesirable effects and carry risks which need to be taken into account when choosing a specific treatment. The side effects of some treatments also include the promotion of the cancer. A treatment that has all the benefits of the current treatments but without or with a reduced risk of promoting the development of the cancer would be highly beneficial.

We have now found that certain matrix metalloproteinase inhibitors are effective when used in combination with radiotherapy therapy for the treatment of tumors especially tumors of the brain, breast, larynx, pancreas, skin, tongue, uterine cervix also leukaemia and lymphoma. Further we have found that such matrix metalloproteinase inhibitors may be used in combination with heat shock in combination with additional cytotoxic therapy for the treatment of such tumours.

Accordingly in a first aspect the invention provides a method of treating cancer in a subject in need of such treatment which comprises administering to the subject an effective amount of a matrix metalloproteinase inhibitor in combination with radiotherapy.

Accordingly in a second aspect the invention provides a method of treating tumors in a subject in need of such treatment which comprises administering to the subject an effective amount of a matrix metalloproteinase inhibitor in combination with heat shock and cytotoxic therapy

Preferably the invention provides a method of treating tumors in a subject in need of such treatment which comprises administering to the subject an effective amount of a hydroxamic acid derivative matrix metalloproteinase inhibitor (of the formula I) in combination with
radiotherapy, or
heat shock and cytotoxic therapy.

Hydroxamic acid derivative metalloproteinase inhibitors are well known in the art. A suitable metalloproteniase inhibitor for use in the method of the invention is, for instance, a compound of formula I 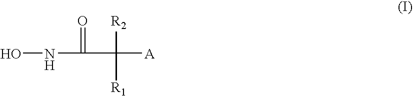
(i) Wherein
A represents substituent of formula II or III; 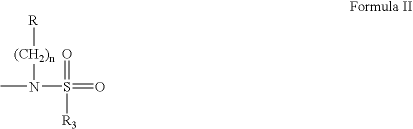
   wherein
R represents hydrogen, lower alkyl, aryl-lower alkyl, aryl, mono- or poly-halo-lower alkyl, cycloalkyl, cycloalkyl-lower alkyl, (oxa or thia)-cycloalkyl, [(oxa or thia)-cycloalkyl]-lower alkyl, hydroxy-lower alkyl, acyloxy-lower alkyl, lower alkoxy-lower alkyl, lower alkyl-(thio, sulfinyl or sulfonyl)-lower alkyl, (amino, mono- or di-lower alkylamino)-lower alkyl, acylamino-lower alkyl, (N-lower alkyl-piperazino or N-aryl-lower alkylpiperazino)-lower alkyl, or (morpholino, thiomorpholino, piperidino, pyrrolidino, piperidyl or N-lower alkylpiperidyl)-lower alkyl;
R₃ represents aryl that may be unsubstituted or substituted by R₄ and R₅;
R₄ and R₅ independently represent hydrogen, lower alkyl, lower alkoxy, halogen, hydroxy, acyloxy, lower alkoxy-lower alkoxy, trifluromethyl or cyano, oxy-C₂-C₃-alkylene, 1- or 2-napthyl; or R₄ and R₅ together on adjacent carbon atoms represent lower alkylenedioxy;
n represents an integer from 1 to 5; 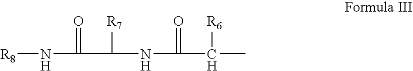
   wherein
R₆ is C₃₋₁₂ alkyl, C₃₋₁₂ alkenyl, C₃₋₇ (optionally hydroxy-, C₁₋₆ alkoxy-, amino-, or C₁₋₆ alkylamino-substituted) cycloalkyl, C₅₋₁₄ aryl, or C₅₋₁₄ aryl(C₁₋₆ alkyl), wherein aryl groups are optionally substituted by hydroxy-, C₁₋₆ alkyl-, C₁₋₆ alkoxy-, amino-, halo- or cyano-;
R₇ is C₁₋₁₀ (optionally hydroxy- or C₁₋₆alkoxy-amino-, C₁₋₆ alkylamino-, thiol-, C₁₋₆ alkylmercapto- or protected hydroxy-, amino- or thiol-substituted) alkyl, C₆₋₁₄ (optionally hydroxy-, C₆₋₁₄aryloxy-, or C₁₋₆alkoxy-, amino-, C₁₋₆ alkylamino-, halo-, or cyano-substituted) aryl, or indolylmethyl;
R₈ is methyl, pyridyl, or a substituent of formula X—Y— wherein X is morpholino, pyridyl or aryl, and Y is C₁₋₁₂alkylene in which up to four of the methylene (—CH₂—) units are optionally replaced with —CO—, —NH—, —SO₂— or —O—;
R₁ is hydrogen, lower alkyl, aryl, aryl-lower alkyl, mono- or poly-halo-lower alkyl, cycloalkyl, cycloalkyl-lower alkyl, cycloalkyl-cycloalkyl, aryl-lower alkyl-lower cycloalkyl, lower alkyl-cycloalkyl, lower alkoxy-lower alkyl-cycloalkyl, aryl-cycloalkyl, cycloalkyl-lower alkyl-cycloalkyl, halo-lower alkyl-cycloalkyl, hydroxy-lower alkyl, acyloxy-lower alkyl, lower alkoxy-lower alkyl, aryl-lower alkoxy-lower alkyl, lower alkyl-(thio, sulfinyl or sulfonyl)-lower alkyl, (amino, mono- or di-lower alkylamino)-lower alkyl, (N-lower alkyl-piperazino or N-aryl-lower alkylpiperazino)-lower alkyl, (morpholono, thiomorpholino, piperidino, pyrrolidino, piperidyl or N-lower alkylpiperidyl)-lower alkyl, acylamino-lower alkyl, piperidyl, N-lower alkylpiperidyl or a substituent of formula IV
   D-(O—(CR₉H)_{z})ₘ—O—CH₂ Formula IV
   wherein
z is 1,2, 3 or 4;
m is 0, 1, 2 or 3;
each R₉ is independently H, C₁₋₁₀ (optionally hydroxy-, C₁₋₆ alkoxy-, amino-, C₁₋₆ alkylamino-, thiol-, C₁₋₆ alkylmercapto- or protected hydroxy, amino or thiol substituted) alkyl, C₂₋₆ alkenyl, C₆₋₁₄ (optionally hydroxy-, C₁₋₆ alkoxy-, amino-, C₁₋₆ alkylamino-, halo- or cyano-substituted) aryl, or C₆₋₁₄ (aryl) C₁₋₆alkyl;
D is hydrogen, C₁₋₁₀ alkyl, C₆₋₁₄ aryl, C₆₋₁₄ aryl(C₁₋₆ alkyl), (C₆₋₁₄ aryl)carbonyl, or (C₁₋₁₀ alkyl)carbonyl;
R₂ is hydrogen or lower alkyl;
   or (ii) wherein
R (of formula II under (a)) and R₁ together with the chain to which they are attached form a 1,2,3,4-tetrahydro-isoquinoline, piperidine, oxazolidine, thiazolidine or pyrrolidine ring, each unsubstituted or substituted by lower alkyl; and
R₃ and R₂ have meaning as defined under (i);
   or (iii) wherein
R₁ and R₂ together with the carbon atom to which they are attached form a ring system selected from lower cycloalkane which is unsubstituted or substituted by lower alkyl′ oxa-cyclohexane, thia-cyclohexane, indane, tetralin, piperidine or piperidine substituted on nitrogen by acyl, lower alkyl, aryl-lower alkyl, (carboxy, esterified or amidated carboxy)-lower alkyl or by lower alkylsulfonyl; and
R₃ and R meaning as defined under (i);
   or a pharmaceutically acceptable prodrug derivative thereof; or a pharmaceutically acceptable salt thereof.

Further the invention provides the use of a hydroxamic acid derivative metalloproteinase inhibitor, for instance a compound of formula I (or pharmaceutically acceptable salt or prodrug ester thereof) for the preparation of a medicament for use in combination with
radiotherapy, or
heat shock and cytotoxic therapy,
   in the treatment of tumors.

In a further aspect the invention provides use of a hydroxamic acid derivative metalloproteinase inhibitor, for instance a compound of formula I (or pharmaceutically acceptable salt or prodrug ester thereof) in combination with
a) radiotherapy, or
b) heat shock and cytotoxic therapy,
   for the treatment of tumors.

In yet further aspect the invention provides a hydroxamic acid derivative matrix metalloproteinase inhibiting agent comprising, for instance a compound of formula I (or pharmaceutically acceptable salt or prodrug ester thereof) as active ingredient for use in combination with
radiotherapy, or
heat shock and cytotoxic therapy,
   for the treatment of tumors which involve heat shock induced MMP expression, especially MMP-3 expression.

In a yet further aspect the invention provides a package comprising a hydroxamic acid derivative metalloproteinase inhibitor, for instance a compound of formula I (or pharmaceutically acceptable salt or prodrug ester thereof) together with instructions for the use in combination with
radiotherapy, or
heat shock and cytotoxic therapy,
   in the treatment of tumors.

The invention may be used for the treatment of any tumor which is susceptible to treatment by cytotoxic therapy, including the treatment of solid tumours, carcinoma, adenocarcinoma. For example the invention may be used in the treatment of tumors of the brain, breast, larynx, skin, tongue, uterine cervix and also leukaemia and lymphoma, especially pancreatic tumors.

Above and elsewhere in the present description the following terms have the meanings given below:

The term “lower” referred to above and hereinafter in connection with organic radicals or compounds respectively defines a compound or radical which may be branched or unbranched with up to and including 7, preferably up to and including 4 carbon atoms.

A lower alkyl group is branched or unbranched and contains 1 to 7 carbon atoms, preferably 1-4 carbon atoms. Lower alkyl represents, for example, methyl, ethyl, propyl, butyl, isopropyl or isobutyl.

A lower alkoxy (or alkyloxy) group preferably contains 1-7 carbon atoms, advantageously 1-6 carbon atoms, and represents for example methoxy, ethoxy, propoxy, isopropoxy, isobutoxy, preferably methoxy. Lower alkoxy includes cycloalkyloxy and cycloalkyl-lower alkyloxy.

Halogen (halo) preferably represents chloro or fluoro but may also be bromo or iodo.

Aryl represents carbocyclic or heterocyclic aryl including biaryl.

Carbocyclic aryl represents monocyclic, bicyclic or tricyclic aryl, for example phenyl or phenyl mono-, di- or tri-substituted by one, two or three radicals selected from lower alkyl, lower alkoxy, hydroxy, halogen, cyano, trifluoromethyl, lower alkylenedioxy, and oxy-C2-C3-alkylene; or 1- or 2-naphthyl. Lower lkylene is a divalent substituent attached to two adjacent carbon atoms of phenyl, e.g. methylenedioxy or ethylenedioxy. Oxy-C2-C3-alkylene is also a divalent substituent attached to two adjacent carbon atoms pf phenyl, e.g. oxyethylene or oxypropylene, An example for oxy-C2-C3-alkylene-phenyl is 2,3-dihydrobenzofuran-5-yl.

Heterocyclic aryl represents monocyclic or bicyclic heteroaryl, for example pyridyl, indolyl, quinoxalinyl, quinolyl, isoquinolyl, benzothienyl, benzofuranyl, benzopyranyl, benzothiopyranyl, furanyl, pyrrolyl, thiazolyl, oxazolyl, isoxazolyl, triazolyl, tetrazolyl, pyrazolyl, imidazolyl, thienyl, or any said radical substituted, especially mono- or di-substituted, by lower alkyl or halogen. Pyridyl represents 2-, 3- or 4-pyridyl, advantageously 2- or 3-pyridyl. Thienyl represents 2- or 3-thienyl, advantageously 2-thienyl. Quinolyl represents 2-, 3- or 4-quinolyl, advantageously 2-quinolyl. Isoquinolyl represents preferably 1-, 3- or 4-isoquinolyl. Benzopyranyl, benzothiopyranyl represent preferably 3-benzopyranyl or 3-benzothiopyranyl, respectively. Thiazolyl represents preferably 2- or 4-thiazolyl, advantageously 4-thiazolyl. Triazolyl is preferably 1-, 2- or 5-(1,2,4-triazolyl). Tetrazolyl is preferably 5-tetrazolyl. Imidazolyl is preferably 4-imidazolyl.

Biaryl is preferably carbocyclic biaryl, e.g biphenyl, namely 2, 3 or 4-biphenyl, advantageously 4-biphenyl, each optionally substituted by e.g. lower alkyl, lower alkoxy, halogen, trifluoromethyl or cyano.

Cycloalkyl represents a saturated cyclic hydrocarbon optionally substituted by lower alkyl which contains 3 to 8 ring carbons and is advantageously cyclopropyl, cyclobutyl, cyclopentyl, or cyclohexyl optionally substituted as hereinbefore defined; cycloalkyl includes heterocyclyl.

Heterocyclyl represents a saturated cyclic hydrocarbon containing one or more, preferably 1 or 2, hetero atoms selected from O, N or S, and preferably from 3 to 10, more preferably 5 to 8, ring atoms; for example, tetrahydrofuranyl, tetrahydrothienyl, tetrahydropyrrolyl, piperidinyl, piperazinyl or morpholino; all of which may be optionally substituted, for instance as hereinbefore defined.

Amino may be optionally substituted, e.g. by lower alkyl.

Aryl-lower alkyl represents preferably (carbocyclic aryl or heterocylic aryl)-lower alkyl.

Carbocyclic aryl-lower alkyl preferably represents aryl-straight chain or -branched C₁₋₄-alkyl in which carbocyclic aryl has meaning as defined above, e.g. benzyl or phenyl-(ethyl, propyl or butyl), each unsubstituted or substituted preferably on the phenyl ring as hereinbefore defined for carbocyclic aryl above.

Heterocyclic aryl-lower alkyl represents preferably straight chain or branched heterocyclic aryl-C₁₋₇-alkyl in which heterocyclic aryl has meaning as defined above.

Cycloalkyl-lower alkyl represents e.g. (cyclopropyl- or cyclobutyl)-(methyl or ethyl).

Combination refers to all combinations, of a MMP inhibitor of formula I,
radiotherapy, or
heat shock and cytotoxic therapy,
   such that there is an effect which would not be obtained if the MMP inhibitor of formula I is administered without prior, simultaneous or subsequent
radiotherapy, or
heat shock and cytotoxic therapy.

The radiotherapy or heat shock and cytotoxic therapy can be continuous, sequential or sporadic. Preferably the effect obtained is such as would not be obtained if there is cytotoxic therapy without prior, simultaneous or subsequent radiotherapy or heat shock therapy with administration of a MMP inhibitor of formula I. Radiotherapy, heat shock or administration of MMP inhibitor of formula I may be continuous, sequential or sporadic

Preferably combination refers to all combinations, of a MMP inhibitor of formula I, radiotherapy or heat shock and cytotoxic therapy, such that there is an effect on MMP expression or tumour invasion potential which would not be obtained if
a) The MMP inhibitor is administered without prior, simultaneous or subsequent radiotherapy or heat shock and prior, simultaneous or subsequent cytotoxic therapy, wherein radiotherapy or heat shock and cytotoxic therapy can be continuous, sequential or sporadic, and
   wherein cytotoxic therapy can be continuous, sequential or sporadic;
b) there is cytotoxic therapy without prior, simultaneous or subsequent administration of radiotherapy or heat shock and without prior, simultaneous or subsequent administration of a MMP inhibitor, wherein administration of radiotherapy or heat shock and MMP inhibitor can be continuous, sequential or sporadic.
c) There is radiotherapy or heat shock without prior, simultaneous or subsequent cytotoxic therapy, and without prior, simultaneous or subsequent administration of a matrix metalloproteinase inhibitor, and wherein administration of heat shock and matrix metalloproteinase inhibitor can be independently continuous, sequential or sporadic and Cytotoxic therapy refers to a therapy or combination of therapies which causes cell damage or death. For example those therapies which are known for treating cancer for example Biological therapy (e.g. Interferon, Interleukin-2), Chemotherapy, Chemotherapy drugs (e.g Actinomycin D, Adriamycin, Altretamine, Asparaginase, Bleomycin, Busulphan, Capecitabine, Carboplatin, Carmustine, Chlorambucil, Cisplatin, Cyclophosphamide, Cytarabine, Dacarbazine, Daunorubicin, Doxorubicin, Epirubicin, Etoposide, Fludarabine, Fluorouracil, Gemcitabine, Hydroxyurea, Idarubicin, Ifosfamide, Irinotecan, Liposomal Doxorubicin, Lomustine, Melphalan, Mercaptopurine, Methotrexate, Mitomycin, Mitozantrone, Oxaliplatin, Procarbazine, Steroids, Streptozocin, Taxol, Taxotere, Taxotere—the TACT trial, Tamozolomide, Thioguanine, Thiotepa, Tomudex, Topotecan, Treosulfan, UFT (Uracil-tegafur), Vinblastine, Vincristine, Vindesine, Vinorelbine), Combination chemotherapy regimes (e.g. Mayo regime, de Gramont regime, Irinotecan with de Gramont regime, ECF regime, ECF regime, Paclitaxel (Taxol) and Carboplatin, CHOP regime, AC regime, CMF regime, EC regime, MM regime, MMM regime, ECF regime), monoclonal antibodies (e.g. Rituximab, Tositumomab, Tratuxumab), Imatinib, photodynamic therapy, radiotherapy. Preferably cytotoxic therapy refers to radiotherapy.

Thus in a particularly preferred embodiment the invention provides a method of treating cancer in a subject in need of such treatment which comprises administering to the subject an effective amount of a matrix metalloproteinase inhibitor in combination with radiotherapy and heat shock treatment.

Heat shock refers to any method of causing a heat shock response by a cell or cells in a tumor or within the area of a tumor. Heat shock may be administered to the whole body, part of the body or locally to the tumor and may be caused by external or internal means, for example heating rods, microwaves, radiofrequences, ultrasound, thermal blankets, thermal baths, lasers, inducing fever e.g administration of a pyrogen, etc.

Radiotherapy may may comprise any of the forms of radiation therapy used or proposed for use in treatment of cancers, including tumours. Thus, for example, gamma radiation may be used or X-ray radiation or any of the other forms of radiation customarily used for cancer treatment.

The term “tumor” is intended to mean malignant tumors and benign tumors in particular cancerous tumors for example cancers of the brain, breast, larynx, pancreas, skin, tongue, uterine cervix also leukaemia and lymphoma.

Preferred embodiments provide a method of treating tumor which can be treated with cytotoxic therapy in a subject in need of such treatment which comprises cytotoxic therapy and heat shock in combination with administering to the subject an effective amount of;
a) Compound of formula V 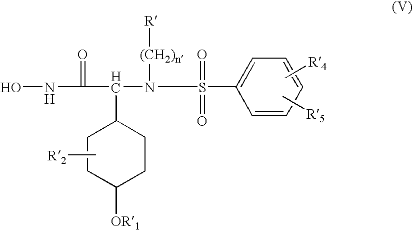
   wherein
R′ represents aryl;
R′₁ represents lower alkyl, cycloalkyl, aryl-lower alkyl, lower alkoxy-lower alkyl, aryl, cycloalkyl-lower alkyl or halogen-lower alkyl;
R′₂ represents hydrogen or lower alkyl;
R′₄ and R′₅ represent independently hydrogen, lower alkyl, lower alkoxy, halogen, hydroxy, acyloxy, lower alkoxy-lower alkoxy, trifluoromethyl or cyano; or R′₄ and R′₅ together on adjacent carbon atoms represent lower alkylenedioxy;
n′ represents an integer from 1 to 5;
   or a pharmaceutically acceptable prodrug derivative thereof; or a pharmaceutically acceptable salt thereof;
b) Compound of formula VI 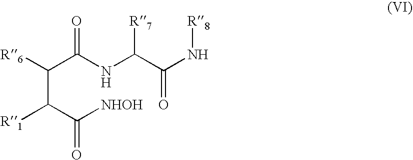
   wherein
R″₁ is a substituent of Formula IV″:
   D″-(O—(CR″₉H)_{z″})_{m″}—O—CH₂— Formula IV″
   wherein
z″ is 1, 2, 3 or 4, preferably 2;
m″ is 0, 1, 2 or 3;
each R″₉ is independently H, C₁₋₁₀ (optionally hydroxy-, C₁₋₆ alkoxy-, amino-, C₁₋₆ alkylamino-, thiol-, C₁₋₆ alkylmercapto- or protected hydroxy, amino or thiol substituted) alkyl, C₂₋₆ alkenyl, C₆₋₁₄ (optionally hydroxy-, C₁₋₆ alkoxy-, amino-, C₁₋₄ alkylamino-, halo- or cyano-substituted) aryl, or C₆₋₁₄ (aryl) C₁₋₆ alkyl; preferably H, phenyl, benzyl or C₁₋₅ alkyl;
D″ is hydrogen, C₁₋₁₀ alkyl, C₆₋₁₄ aryl, C₆₋₁₄ aryl(C₁₋₆ alkyl), (C₆₋₁₄ aryl)carbonyl, or (C₁₋₁₀ alkyl)carbonyl; preferably hydrogen, C₁₋₆ alkyl (e.g., methyl or cyclohexyl), phenyl or benzyl;
R″₆ is C₃₋₁₂ alkyl, C₃₋₁₂ alkenyl, C₃₋₇ (optionally hydroxy-, C₁₋₆ alkoxy-, amino-, or C₁₋₆ alkylamino-substituted) cycloalkyl, C₅₋₁₄ aryl, or C₅₋₁₄ aryl(C₁₋₆ alkyl), wherein aryl groups are optionally substituted by hydroxy-, C₁₋₆ alkyl-, C₁₋₆ alkoxy-, amino-, halo- or cyano-; preferably phenyl, 4-methylphenyl, cyclohexyl or isobutyl;
R″₇ is C₁₋₁₀ (optionally hydroxy- or C₁₋₆alkoxy-amino-, C₁₋₆ alkylamino-, thiol-, C₁₋₆ alkylmercapto- or protected hydroxy-, amino- or thiol-substituted) alkyl (e.g., t-butyl, or cyclohexylmethyl), C₆₋₁₄ (optionally hydroxy-, C₆₋₁₄aryloxy-, or C₁₋₆alkoxy-, amino-, C₁₋₆ alkylamino-, halo-, or cyano-substituted) aryl (e.g., benzyl, p-methoxybenzyl, p-benzyloxybenzyl), or indolylmethyl (e.g., 2-indolylmethyl); preferably benzyl or t-butyl;
R″₈ is methyl, pyridyl, or a substituent of formula X″—Y″— wherein X″ is morpholino, pyridyl or aryl (preferably morpholino), and Y″ is C₁₋₁₂alkylene in which up to four of the methylene (—CH₂—) units are optionally replaced with —CO—, —NH—, —SO₂— or —O—; for example methyl, 2-pyridyl, morpholinocarbonylmethyl, 5-(morpholino)pentyl, or 5-(morpholinocarbonyl)pentyl;
   or a pharmaceutically acceptable prodrug derivative thereof; or a pharmaceutically acceptable salt thereof;
c) Compound of formula VII 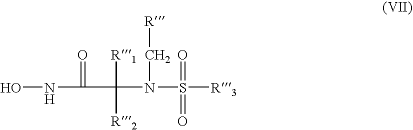
   (i′) wherein
R′″ represents hydrogen, lower alkyl, aryl-lower alkyl, aryl, mono- or poly-halo-lower alkyl, cycloalkyl, cycloalkyl-lower alkyl, (oxa or thia)-cycloalkyl, [(oxa or thia)-cycloalkyl]-lower alkyl, hydroxy-lower alkyl, acyloxy-lower alkyl, lower alkoxy-lower alkyl, lower alkyl-(thio, sulfinyl or sulfonyl)-lower alkyl, (amino, mono- or di-lower alkylamino)-lower alkyl, acylamino-lower alkyl, (N-lower alkyl-piperazino or N-aryl-lower alkylpiperazino)-lower alkyl, or (morpholino, thiomorpholino, piperidino, pyrrolidino, piperidyl or N-lower alkylpiperidyl)-lower alkyl;
R′″₁ is hydrogen, lower alkyl, aryl, aryl-lower alkyl, mono- or poly-halo-lower alkyl, cycloalkyl, cycloalkyl-lower alkyl, hydroxy-lower allyl, acyloxy-lower alkyl, lower alkoxy-lower alkyl, aryl-lower alkoxy-lower alkyl, lower alkyl-(thio, sulfinyl or sulfonyl)-lower alkyl, (amino, mono- or di-lower alkylamino)-lower alkyl, (N-lower alkyl-piperazino or N-aryl-lower alkylpiperazino)-lower alkyl, (morpholono, thiomorpholino, piperidino, pyrrolidino, piperidyl or N-lower alkylpiperidyl)-lower alkyl, acylamino-lower alkyl, piperidyl or N-lower alkylpiperidyl;
R′″₂ is hydrogen or lower alkyl;
R′″₃ represents aryl which may be unsubstituted or substituted by R′″₄ and R′″₅;
   or (ii′) wherein
R′″ and R′″₁ together with the chain to which they are attached from a 1,2,3,4-tetrahydro-isoquinoline, piperidine, oxazolidine, thiazolidine or pyrrolidine ring, each unsubstituted or substituted by lower alkyl; and R′″₂ and R′″₃ have meaning as defined under (i′);
   Or (iii′) wherein
R′″₁ and R′″₂ together with the carbon atom to which they are attached form a ring system selected from lowercycloalkane which is unsubstituted or substituted by lower alkyl′ oxa-cyclohexane, thia-cyclohexane, indane, tetralin, piperidine or piperidine substituted on nitrogen by acyl, lower alkyl, aryl-lower alkyl, (carboxy, esterified or amidated carboxy)-lower alkyl or by lower alkylsulfonyl; and R′″₃ and R′″ meaning as defined under (i′);
   or a pharmaceutically acceptable prodrug derivative thereof; or a pharmaceutically acceptable salt thereof.

Particularly preferred embodiments provide a method of treating cancer which can be treated with radiotherapy in a subject in need of such treatment which comprises radiotherapy and/or heat shock in combination with administering to the subject an effective amount of;
a′) Compound of formula V having the trans configuration with respect to the 1,4-substituents on the cyclohexane ring, particularly those of formula V′ 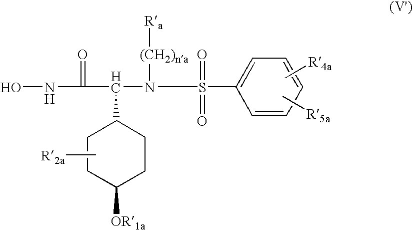
   wherein
R′ₐ represents aryl;
R′₁ₐ represents lower alkyl, cycloalkyl, aryl-lower all or lower alkoxy-lower alkyl;
R′₂ₐ represents hydrogen or lower alkyl;
R′₄ₐ is hydrogen, lower alkoxy or halogen;
R′₅ₐ is hydrogen or lower alkoxy; or
R′₄ₐ and R′₅ₐ together on adjacent carbon atoms represent methylenedioxy; and
n′ₐ is 1-4;
   or a pharmaceutically acceptable prodrug derivative thereof; or a pharmaceutically acceptable salt thereof;
b′) Compound of Formula VI′ 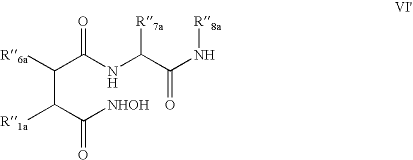
   wherein;
(i) R″₁ₐ is of formula IV′″ or IV″″ (preferably formula IV′″)
   D″-(O—(CH₂)_{z″})_{m″}—O—CH₂— Formula IV′″
   wherein D″, z″ and m″ are as defined above;
   D″-O—(CHR″₉—(CH₂)_{z″})_{m″′}—O—CH₂— Formula IV″″
   wherein D″, z″ and R″₉ are as defined above and m″′is 0, 1 or 2.
   or D″ of formula IV′″is hydrogen, C₁₋₆ alkyl, e.g., methyl or cyclohexyl (e.g., so that R″₁ₐ of formula VI′ is for example hydroxymethyl, cyclohexyloxyethoxymethyl, methoxyethoxyethoxymethyl, or hydroxyethyloxymethyl) or (C₆₋₁₄ aryl)carbonyl, e.g. benzoyl (e.g. so that R″₁ of formula VI is for example benzoyloxymethyl, benzoyloxyethoxyethyl or benzoyloxyethoxyethoxymethyl);
(ii) R″₆ₐ of formula VI′ is cyclohexyl, phenyl, 4-methylphenyl or isobutyl;
(iii) R″₇ₐ of formula VI′ is benzyl or t-butyl; and
(iv) R″₈ₐ of formula VI′ is methyl or morpholinocarbonyl(C₁₋₆)alkyl,
   or a pharmaceutically acceptable prodrug derivative thereof; or a pharmaceutically acceptable salt thereof.

The configuration of the Compounds of formula VI′ is preferably that of Formula VIa: 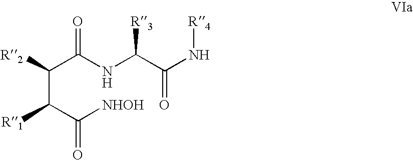
or of Formula VIb: 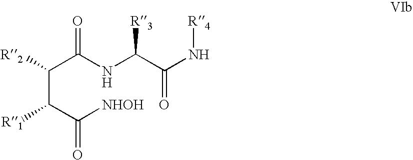
most preferably that of Formula VIa.

Further particularly preferred embodiments provide a method of treating cancer which can be treated with radiotherapy in a subject in need of such treatment which comprises radiotherapy and/or heat shock in combination with administering to the subject an effective amount of:
c′) Compound of formula VII having R₃ represent phenyl which may be unsubstituted or substituted by R′″₄ and R′″₅ herein before defined, particularly those of the formula VII′: 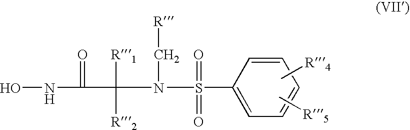
   wherein
R′″ represents hydrogen, lower alkyl, aryl-lower alkyl, aryl, mono- or poly-halo-lower alkyl, cycloalkyl, cycloalkyl-lower alkyl, (oxa or thia)-cycloalkyl, [(oxa or thia)-cycloalkyl]-lower alkyl, hydroxy-lower alkyl, acyloxy-lower alkyl, lower alkoxy-lower alkyl, lower alkyl-(thio, sulfinyl or sulfonyl)-lower alkyl, (amino, mono- or di-lower alkylamino)-lower alkyl, acylamino-lower alkyl, (N-lower alkyl-piperazino or N-aryl-lower alkylpiperazino)-lower alkyl, or (morpholino, thiomorpholino, piperidino, pyrrolidino, piperidyl or N-lower alkylpiperidyl)-lower alkyl;
R′″ ₁ is hydrogen, lower alkyl, aryl, aryl-lower alkyl, mono- or poly-halo-lower alkyl, cycloalkyl, cycloalkyl-lower alkyl, hydroxy-lower alkyl, acyloxy-lower alkyl, lower alkoxy-lower alkyl, lower alkoxy-lower alkyl, lower alkyl-(thio, sulfinyl or sulfonyl)-lower alkyl, (amino, mono- or di-lower alkylamino)-lower alkyl, (N-lower alkyl-piperazino or N-aryl-lower alkylpiperazino)-lower alkyl, (morpholino, thiomorpholino, piperidino, pyrrolidino, piperidyl or N-lower alkylpiperidyl)-lower alkyl, piperidyl, N-lower alkylpiperidyl or acylamino-lower alkyl represented by R′″₁₀—CONH-lower alkyl;
R′″₂ is hydrogen;
R′″₁₀ in R′″₁₀—CONH-lower alkyl is lower alkyl, aryl, di-lower alkylamino, N-lower alkylpiperazino, morpholino, thiomorpholino, piperidino, pyrrolidino, N-alkylpiperidyl, or (di-lower alkylamino, N-lower alkylpiperazino, morpholino, thiomorpholino, piperidino, pyrrolidino, pyridyl or N-lower alkylpiperidyl)-lower alkyl;
R′″₄ is hydrogen, lower alkoxy, hydroxy, aryl-lower alkoxy, lower alkylthio or aryl-lower alkylthio, lower alkyloxy-lower alkoxy, halogen, trifluoromethyl, lower alkyl, nitro or cyano;
R′″₅ is hydrogen, lower alkyl or halogen;
or R′″₄ and R′″₅ together on adjacent carbon atoms represent methylenedioxy, ethylenedioxy, oxyethylene or oxypropylene;
   or a pharmaceutically acceptable prodrug derivative thereof; or a pharmaceutically acceptable salt thereof.

Further and most preferred embodiments provide a method of treating a tumour in a subject in need of such treatment which comprises administering to the subject an effective amount of a pharmaceutical composition for use in combination with heat shock and/or radiotherapy wherein said pharmaceutical composition comprises
a″) compound of formula VII″ 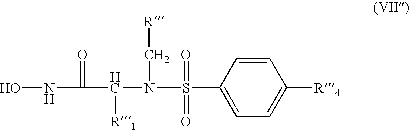
   wherein;
R′″ represents lower alkyl, aryl, trifluromethyl, cycloalkyl, (oxa or thia)-cycloalkyl;
R′″₁ is hydrogen, lower alkyl, aryl, aryl-lower alkyl, lower alkoxy-lower alkyl, lower alkyl-(thio, sulfinyl or sulfonyl)-lower alkyl, di-lower alkylamino-lower alkyl, (N-lower alkyl-piperazino, morpholino, thiomorpholino, piperidino, pyrrolidino)-lower alkyl or R′″₁₀—CONH-lower alkyl;
R′″₁₀ in R″′₁₀—CONH-lower alkyl is lower alkyl, aryl, di-lower alkylamino, N-lower alkylpiperazino, morpholino, thiomorpholino, piperidino, pyrrolidino, N-alkylpiperidyl, or (di-lower alkylamino, N-lower alkylpiperazino, morpholino, thiomorpholino, piperidino, pyrrolidino or N-lower alkylpiperidyl)-lower alkyl;
R′″₄ is hydrogen, lower alkoxy, aryl-lower alkoxy;
   or a pharmaceutically acceptable prodrug derivative thereof; or a pharmaceutically acceptable salt thereof;
b″) Compound of formula I, V, VI, VII, V′, VI′, VII′, VIa, VIb or VII″ that is a matrix metalloproteinase inhibitor,
   or a pharmaceutically acceptable prodrug derivative thereof, or a pharmaceutically acceptable salt thereof; or
c″) one of the compounds disclosed in published international patent applications Nos. WO 98/14424, WO 97/22587 and EP 606046, in particular the compound N-hydroxy-2(R)-[[4-methoxyphenylsulfonyl](3-picolyl)amino]-3-methyl-butaneamide hydrochloride)monohydrate;
   or a pharmaceutically acceptable prodrug derivative thereof, or a pharmaceutically acceptable salt thereof.

Compounds of formula I, II, III, IV, V, VI an VII and their synthesis are described in published international patent applications Nos. WO 98/14424, WO 97/22587 and EP 606046, the teachings of which are incorporated herein by reference.

The agents of the invention, i.e. the MMP inhibitors of formula I and pharmaceutically acceptable salts and prodrug derivatives, are preferably used in the form of pharmaceutical preparations that contain the relevant therapeutically effective amount of active ingredient optionally together with or in admixture with inorganic or organic, solid or liquid, pharmaceutically acceptable carriers which are suitable for administration.

The MMP inhibitor pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic), or compositions for topical administration,

Preferably, the MMP inhibitor pharmaceutical compositions are adapted to oral administration.

The particular mode of administration and the dosage may be selected by the attending physician taking into account the particulars of the patient, especially age, weight, life style, activity level, etc.

The dosage of the Agents of the invention may depend on various factors, such as effectiveness and duration of action of the active ingredient, mode of administration, and/or sex, age, weight and individual condition of the subject to be treated.

The agents of the invention are useful in the manufacture of pharmaceutical compositions comprising an effective amount thereof in conjunction or admixture with excipients or carriers suitable for either enteral or parenteral application. In addition, they may also contain other therapeutically valuable substances. Said compositions are prepared according to conventional mixing, granulating or coating methods, respectively, and contain about 0.1 to 75%, preferably about 1 to 50%, of the active ingredient.

Parenteral formulations are especially injectable fluids that are effective in various manners, such as intravenously, intramuscularly, intraperitoneally, intranasally, intradermally or subcutaneously. Such fluids are preferably aqueous isotonic solutions or suspensions that can be prepared before use, for example from lyophilised preparations that contain the active ingredient alone or together with a pharmaceutically acceptable carrier. The pharmaceutical preparations be sterilised and/or contain adjuncts, for example preservatives, stabilisers, wetting agents and/or emulsifiers, solubilisers, salts for regulating the osmotic pressure and/or buffers.

Suitable oral forms are tablets and gelatin capsules comprising the active ingredient together with a) diluents, e.g. lactose, dextrose, sucrose, mannitol, sorbitol, cellulose and/or glycine; b) lubricants, e.g. silica, talcum, stearic acid, its magnesium or calcium salt and/or polyethyleneglycol; for tablets also c) binders e.g. magnesium aluminium silicate, starch paste, gelatin, tragacanth, methylcellulose, sodium carboxymethylcellulose and or polyvinylpyrrolidone; if desired d) disintegrants, e.g. starches, agar, alginic acid, or it's sodium salt, or effervescent mixtures; and/or e) adsorbents, colorants, flavours and sweeteners. Tablets may be either film coated or enteric coated according to methods known in the art.

Suitable formulations for transdermal application include an effective amount of a compound of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Suitable formulations for topical application, e.g. to the skin and eyes, include aqueous solutions, suspensions, ointments, creams, gels, or sprayable formulations, for example, for delivery by aerosol or the like. Such topical formulations typically contain from about 0.1 up to about 50% by weight, preferably from about 1 up to about 20% by weight, of MMP inhibitor.

The following examples are intended to illustrate the invention and are not to be construed as being limitations thereon.

### EXAMPLES

### Example 1

Tablets each containing 50 mg of N-hydroxy-2 (R)-[[4-methoxyphenylsulfonyl](3-picolyl)-amino]-3-methylbutanamide hydrochloride can be prepared as follows:

Composition (10,000 tablets)

| | | |
|---|---|---|
| Active ingredient | 500.0 | g |
| Lactose | 500.0 | g |
| Potato starch | 325.0 | g |
| Gelatin | 8.0 | g |
| Talc | 60.0 | g |
| Magnesium stearate | 10.0 | g |
| Silicon dioxide (finely divided) | 20.0 | g |

| | |
|---|---|
| Ethanol | q.s |

The active ingredient is mixed with the lactose and 292 g of potato starch, and the mixture is moistened with an ethanolic solution of the gelatin and granulated through a sieve. After the granules have dried, the remainder of the potato starch, the magnesium stearate and the silicon dioxide are admixed and the mixture compressed to give tablets each weighing 145.0 mg and containing 50.0 mg of active ingredient, which can, if desired, be provided with breaking grooves to enable the dosage to be more freely adjusted.

### Example 2

Preparation of 3000 capsules each containing 25 mg of the active ingredient, for example, N-hydroxy-2 (R)-[[4-methoxyphenylsulfonyl](3-picolyl)-amino]-3-methylbutanamide hydrochloride:

| Active ingredient | 75.0 | g |
|---|---|---|
| Lactose | 750.0 | g |
| Avicel PH 102 | 325.0 | |
| (microcrystalline cellulose) | | |
| Polyplasdone XL | 30.0 | g |
| (polyvinylpyrrolidone) | | |
| Purified water | q.s | |
| Magnesium stearate | 9.0 | g |

The active ingredient is passed through a No. 30 hand screen.

The active ingredient, lactose, Avicel PH 102 and Polyplasdone XL are blended for 15 minutes in a mixer. The blend is granulated with sufficient water (about 500 mL), dried in an oven at 35° C. overnight, and passed through a No. 20 screen.

Magnesium stearate is padded through a No. 20 screen, added to the granulation mixture, and the mixture is blended for 5 minutes in a mixer. The blend is encapsulated in No. 0 hard gelatin capsules each containing and amount of the blend equivalent to 25 mg of the active ingredient.

### Example 3

### Radiotherapy Example

Materials and Methods

Cell Culture and Reagents

Three human pancreatic cancer cell lines are used in this study. Panc-1 and Suit-2 are generously provided by Dr. Iguchi (National Kyushu Cancer Center, Fukuoka, Japan), Hs766T is obtained from American type culture collection (Rockville, Md.). Cells are maintained in Dulbecco's modified Eagle's medium (DMEM, Sigma Chemical Co. St. Louis, Mo., USA) supplemented with 10% fetal bovine serum (FBS), streptomycin (100 μg/ml), and penicillin (100 U/ml) at 37° C. with humidified 90% air and 10% CO₂. The number of cells is counted with a particle distribution counter, CDA500 (Sysmex, Kobe, Japan). The MMP inhibitor N-hydroxy-2(R)-[[4-methoxyphenylsulfonyl](3-picolyl)amino]-3-methyl-butaneamide hydrochloride)monohydrate, is kindly provided by Novartis Pharma, K.K., Japan.

Irradiation

The cells are irradiated with doses of 3, 5, or 10 Gy at room temperature using a ¹³⁷Cs source (Gamma Cell 40, Atomic Energy of Canada Ltd., Ontario, Canada) delivering 1.0 Gy/min.

Cell Proliferation Assay

Cell proliferation is evaluated by measuring the fluorescence intensity of propidium iodide (PI) as described previously by Zhang et al. (Cancer Lett., 142: 129-137, 1999) with minor modifications. Briefly, cells are seeded in 24-well plates at a density of 3×10⁴ cells per well. After overnight cultivation, cells are irradiated and cultured for 4 days. PI (30 μM) and digitonin (600 μl) are added to each well to label all nuclei of the cells with PI. Fluorescence intensity corresponding to total cells in each well is measured by a multi-well plate-reader, CYTOFLUOR II (PerSeptive Biosystems Inc., Framingham, Mass., USA) with 530-nm excitation and 645-nm emission filters. The cell proliferation rate is calculated as the proportion of fluorescence intensity of each well at the time point indicated in the text to that at the day of irradiation.

Migration Assay

Migration of pancreatic cancer cells through 8 μM pores is assessed using the Transwell cell culture chamber (6.5 mm diameter, Corning Costar, Tokyo, Japan) as described by sato et al and Maehara et al (Cancer, 91: 496-504, 2001; Br. J. Cancer, 84: 864-873, 2001). Cells at a density of 1×10⁴ are seeded in the upper chambers with 100 μl of medium supplemented with 10% FBS. Same media of 600 μl are placed in the lower wells. After seeding, the cells are subjected to irradiation and then cultured for 24 h. The filter membranes are removed and fixed with 70% ethanol and stained with hematoxylin and eosin (H&E). The number of cells that had migrated to the lower surface of the filter membrane is counted in five random fields under a light microscope.

Matrigel Invasion Assay

Invasion of pancreatic cancer cells is measured by the invasion of cells through Matrigel-coated transwell inserts (Becton Dickinson, Franklin Lakes, N.J., USA) (Sato et al and Maehara et al ibid).

Briefly, transwell inserts with 8 μm pore are coated with Matrigel (40 μg/well, Becton Dickinson, Bedford, Mass., USA). Five hundred μl of cell suspension (1×10⁵/ml) are added to the upper chambers. Same media of 750 μl are placed in the lower wells. Thereafter, the cells are irradiated and incubated for 24 h. Cells that have invaded to lower surface of the Matrigel-coated membrane are fixed with 70% ethanol, stained with H&E, and counted in five random fields under a light microscope.

Gelatin Zymography

The conditioned medium either from non-irradiated or irradiated Panc-1 cells is concentrated to 10-fold with Centricon-10 (Amico, Beverly, Mass., USA). Samples are added to each lane and subjected to 10% SDS-polyacrylamide gel electrophoresis, using 10% polyacrylamide gel containing 1 mg/ml gelatin. After electrophoresis, the gel is washed in 2.5% Triton X-100, and incubated in 50 mM Tris-HCl buffer (pH8.0) containing 0.5 mM CaCl₂ and 1 mM ZnCl₂ for 20 hr at 37° C. The gel is stained with 1% Coomassie Brilliant Blue R-250 and destained with destaining buffer (5% acetic acid and 10% methanol).

Western Blotting

The proteins (80 μg/lane) from the soluble fraction of Panc-1 cells are fractionated by 10% SDS-polyacrylamide gel electrophoresis and transferred to a polyvinylidene difluoride (PVDF) membrane (Millipore, Bedford, Mass.). The membrane is incubated with 1:500 dilutions of polyclonal antibody for human uPA (urokinase-type plasminogen activator, Santa Cruz Biotechnology, Calif., USA), and then probed with anti-goat IgG conjugated with horseradish peroxides (Santa Cruz Biotechnology, Calif., USA). Immunoblots are detected by the enhanced chemiluminescence (Amersham International, Buckinghamshire, UK).

Statistical Analysis

Statistical analyses are performed by using ANOVA and unpaired Student's t test. All statistics are performed on two-sided test. P<0.05 is considered as significant. Each experiment is repeated at least three times.

Results

Irradiation Inhibits Proliferation of Pancreatic Cancer Cells

First, we examine the proliferation of pancreatic cancer cells after irradiation. Irradiation suppressed the proliferation of Panc-1 cells in a dose-dependent manner, and an almost complete inhibition is observed at a dose of 10 Gy. Similar results are obtained in Suit-2 at the same dose range. In Hs766T cells, however, while dose reached to 5 Gy, radiation had already entirely inhibited the cell growth.

Irradiation Promotes Invasive Potential but Inhibits Migration Ability in a Subset of Pancreatic Cancer Cells

To determine the effect of radiation on cell motility, we analyse the migration of human pancreatic cancer cells before and after irradiation using the Transwell cell migration assay. Compared with untreated controls, Panc-1 and Suit-2 cells irradiated at doses of 3, 5, and 10 Gy show significantly lower numbers of migrated cells. There is no significant change in migration potential after irradiation in Hs766T cells, which show a relatively low basal migration activity.

We next examine changes in the invasive potentials of pancreatic cancer cells after irradiation using the Matrigel invasion assay. In contrast to the decline in migration ability, invasive potentials in both Panc-1 and Suit-2 cells are significantly increased after irradiation at doses of 3, 5, and 10 Gy. This increase in invasive potential appears to be dose-dependent. Remarkably, the average number of invaded cells in Panc-1 is increased by more than 2-fold after irradiation at 10 Gy. We find no significant change in invasive potential in irradiated Hs766T cells.

Irradiation Increases MMP-2 Activity

To determine the role of gelatinases in the radiation-induced changes in invasive potential, we examine MMPs activity in Panc-1 cells before and after irradiation. Cells are incubated 24 h after irradiation and the conditioned medium is subjected to the gelatin zymography. Untreated Panc-1 cells secrete both latent and active forms of MMP-2 (72 kDa and 62 kDa gelatinases). After irradiation, MMP-2 activity of either latent or activated type is significantly increased, thus suggesting that the increased MMP-2 activity may play an important role in the enhanced invasiveness after irradiation.

An MMP Inhibitor Blocks the Radiation-Enhanced Invasion of Pancreatic Cancer Cells

Finally, we test whether a synthetic MMP inhibitor, N-hydroxy-2(R)-[[4 methoxyphenylsulfonyl](3-picolyl)amino]-3-methyl-butaneamide hydrochloride) monohydrate, could prevent the radiation-enhanced invasiveness. N-hydroxy-2(R)-[[4-methoxyphenylsulfonyl](3-picolyl)amino]-3-methyl-butaneamide hydrochloride) monohydrate is added to invasion chambers at final concentrations of 1, 5, and 10 μm just before irradiation. After irradiation at 5 Gy, the number of invaded cells in Panc-1 increase from 14.6 cells/field to 24.4 cells/field, whereas concomitant treatment with N-hydroxy-2(R)-[[4-methoxyphenylsulfonyl](3-picolyl)amino]-3-methyl-butaneamide hydrochloride)monohydrate at concentrations of 5 and 10 μM significantly block the increase in invaded cells after irradiation. Treatment with N-hydroxy-2(R)-[[4-methoxyphenylsulfonyl](3-picolyl)amino]-3-methyl-butaneamide hydrochloride) monohydrate does not affect the growth and viability of Panc-1 cells at concentrations up to 10 μM. Furthermore, gelatin zymography reveals that treatment with N-hydroxy-2(R)-[[4-methoxyphenylsulfonyl](3-picolyl)amino]-3-methyl-butaneamide hydrochloride) monohydrate at 5 μM markedly decreases the active type MMP-2 without affecting the enzymatic activity of latent type MMP-2.

Expression of Urokinase-Type Plasminogen Activator (uPA) Decreases After Irradiation

To determine the possible involvement of uPA in the changes in cell motility after irradiation, we examine the expression of uPA in Panc-1 cells by Western blotting. The uPA expression in cell lysate that represents the constituent portion of uPA is suppressed by irradiation.

### Example 4

### Heat Shock Example

Acquisition of Array Data

For gene expression analysis, HeLa cells seeded in 100 mm petri dish are dipped in a water bath for 1 h at 44° C. (±0.03° C.). RNA is isolated from cells 0, 3, 6 and 12 h after heating. The cells just before heat shock treatment are used as control. Labeled probe is hybridized to a Human 1 cDNA microarray (no. G4100A; Agilent Technologies). The gene expression experiment is repeated two times.

Data analysis

Signal intensities of Cy3 and Cy5 from the 12,814 spots are quantified and analyzed by GenePix (Axon Instruments, Foster City, Calif.). Previously fagged spots by GenePix and 60% of spot pixels with intensities more than one standard deviation above the background pixel intensity are excluded. Residual spot signals are normalized so that median of all signal ratio (Cy3/Cy5) is 1.0. Then extract the genes that showed Cy3/Cy5 signal ratio >2.0 or 0.5<at both two times experiment.

Results

Analysis of Gene Expression Profiles from Data Preprocessing

752 genes are up or down-regulated after heat shock. The temporal pattern of expression for 752 genes is more easily recognized through clustering. Using Fuzzy ART, those genes are separated into 8 clusters. Up-regulated genes at 0 h play an important role in repair of injured cells. “Cluster 1” and “Cluster 2”, containing 53 genes are selected and “Cluster 2” included HSP70 which is well known as heat shock response gene. Among these genes, focus on Matrix metalloproteinase 3 (MMP-3), which is included in “Cluster 2” and conduct next experiment.

Inhibitory Effect Using MMP-3 Inhibitor

MMP-3 inhibitor (no. 444225; CALBIOCHEM) is dissolved in DMSO. The final concentration of MMP-3 Inhibitor in each culture medium is 13 μM. With the same concentration DMSO is used as control. MMP-3 inhibitor is added 1 h before heat shock and dishes are dipped in water bath at 44° C. for 60, 75 and 90 min to make the surviving curve. Surviving cells are counted by trypan blue dye exclusion method after 3 days. MMP-3 Inhibitor induced much more cell death than DMSO. These data indicates that MMP-3 appears to play an important role in restoration of injured cells, and thus inhibition of MMP-3 should inhibit recovery of injured cells.

1. A method of treating cancer in a subject in need of such treatment which comprises radiotherapy, or
cytotoxic therapy in combination with heat shock,
and further comprises administering to the subject an effective amount of a matrix metalloproteinase inhibitor.

2. A method of treating cancer in a subject in need of such treatment which comprises:
radiotherapy,
or cytotoxic therapy in combination with heat shock,
and further comprises administering to the subject an effective amount of a matrix metalloproteinase inhibitor of the formula I 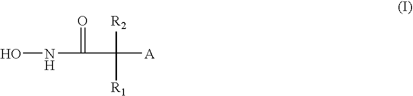
(i) wherein
A represents substituent of formula II or III; 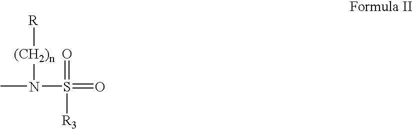
wherein
R represents hydrogen, lower alkyl, aryl-lower alkyl, aryl, mono- or poly-halo-lower alkyl, cycloalkyl, cycloalkyl-lower alkyl, (oxa or thia)-cycloalkyl, [(oxa or thia)-cycloalkyl]-lower alkyl, hydroxy-lower alkyl, acyloxy-lower alkyl, lower alkoxy-lower alkyl, lower alkyl-(thio, sulfinyl or sulfonyl)-lower alkyl, (amino, mono- or all-lower alkylamino)-lower alkyl, acylamino-lower alkyl, (N-lower alkyl-piperazino or N-aryl-lower alkylpiperazino)-lower alkyl, or (morpholino, thiomorpholino, piperidino, pyrrolidino, piperidyl or N-lower alkylpiperidyl)-lower alkyl;
R₃ represents aryl that may be unsubstituted or substituted by R4 and R5;
R₄ or R₅ represents independently hydrogen, lower alkyl, lower alkoxy, halogen, hydroxy, acyloxy, lower alkoxy-lower alkoxy, trifluromethyl or cyano, oxy-C2-C3-alkylene, 1- or 2-napthyl; or R₄ and R₅ together on adjacent carbon atoms represent lower alkylenedioxy;
n represents an integer from 1 to 5; 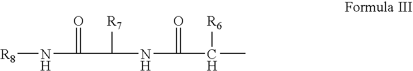
wherein
R₆ is C₃₋₁₂ alkyl, C₃₋₁₂ alkenyl, C₃₋₇ (optionally hydroxy-, C₁₋₆ alkoxy-, amino-, or C₁₋₆ alkylamino-substituted) cycloalkyl, C₅₋₁₄ aryl, or C₅₋₁₄ aryl(C₁₋₆ alkyl), wherein aryl groups are optionally substituted by hydroxy-, C₁ alkyl-, C₁₋₆ alkoxy-, amino-, halo- or cyano-;
R₇ is C₁₋₁₀ (optionally hydroxy- or C₁₋₆ alkoxy-amino-, C₁₋₆ alkylamino-, thiol-, C₁₋₆ alkylmercapto- or protected hydroxy-, amino- or thiol-substituted) alkyl, C₆₋₁₄ (optionally hydroxy-, C₆₋₁₄aryloxy-, or C₁₋₆alkoxy-, amino-, C₁₋₆ alkylamino-, halo-, or cyano-substituted)aryl, or indolylmethyl;
R₈ is methyl, pyridyl, or a substituent of formula X—Y— wherein X is morpholino, pyridyl or aryl, and Y is C₁₋₁₂alkylene in which up to four of the methylene (—CH₂—) units are optionally replaced with —CO—, —NH—, —SO₂— or —O—;
R₁ is hydrogen, lower alkyl, aryl, aryl-lower alkyl, mono- or poly-halo-lower alkyl, cycloalkyl, cycloalkyl-lower alkyl, cycloalkyl-cycloalkyl, aryl-lower alkyl-lower cycloalkyl, lower alkyl-cycloalkyl, lower alkoxy-lower alkyl-cycloalkyl, aryl-cycloalkyl, cycloalkyl-lower alkyl-cycloalkyl, halo-lower alkyl-cycloalkyl, hydroxy-lower alkyl, acyloxy-lower alkyl, lower alkoxy-lower alkyl, aryl-lower alkoxy-lower alkyl, lower alkyl-(thio, sulfinyl or sulfonyl)-lower alkyl, (amino, mono- or di-lower alkylamino)-lower alkyl, (N-lower alkyl-piperazino or N-aryl-lower alkylpiperazino)-lower alkyl, (morpholono, thiomorpholino, piperidino, pyrrolidino, piperidyl or N-lower alkylpiperidyl)-lower alkyl, acylamino-lower alkyl, piperidyl, N-lower alkylpiperidyl or a substituent of formula IV
D-(O—(CR₉H)_{z})ₘ—O—CH₂— Formula IV
wherein
z is 1, 2, 3 or 4;
m is 0, 1, 2 or 3;
each R₉ is
independently H, C₁₋₁₀ (optionally hydroxy-, C₁₋₆ alkoxy-, amino-, C₁₋₆ alkylamino-, thiol-, C₁₋₆ alkylmercapto- or protected hydroxy, amino or thiol substituted) alkyl, C₂₋₆ alkenyl, C₆₋₁₄ (optionally hydroxy-, C₁₋₆ alkoxy-, amino-, C₁₋₆ alkylamino-, halo- or cyano-substituted) aryl, or C₆₋₁₄ (aryl) C₁₋₆alkyl;
D is hydrogen, C₁₋₁₀ alkyl, C₆₋₁₄ aryl, C₆₋₁₄ aryl(C₁₋₁₆ alkyl), (C₆₋₁₄ aryl)carbonyl, or (C₁₋₁₀ alkyl)carbonyl;
R₂ is hydrogen or lower alkyl,
(ii) or wherein
R (of formula II under (a)) and R₁ together with the chain to which they are attached from a 1,2,3,4-tetrahydro-isoquinoline, piperidine, oxazolidine, thiazolidine or pyrrolidine ring, each unsubstituted or substituted by lower alkyl; and
R₃ and R₂ have meaning as defined under (i);
(iii) or wherein
R₁ and R₂ together with the carbon atom to which they are attached form a ring system selected from lowercycloalkane which is unsubstituted or substituted by lower alkyl′ oxa-cyclohexane, thia-cyclohexane, indane, tetralin, piperidine or piperidine substituted on nitrogen by acyl, lower alkyl, aryl-lower alkyl, (carboxy, esterified or amidated carboxy)-lower alkyl or by lower alkylsulfonyl; and
R₃ and R meaning as defined under (i);
or a pharmaceutically acceptable prodrug derivative thereof; or a pharmaceutically acceptable salt thereof.

3. Use of a matrix metalloproteinase inhibitor (or pharmaceutically acceptable salt or prodrug ester thereof) for the preparation of a medicament, for use in combination with a) radiotherapy, or b) heat shock and cytotoxic therapy for the treatment of tumors.

4. Use of a matrix metalloproteinase inhibitor (or pharmaceutically acceptable salt or prodrug ester thereof) in combination with
a) radiotherapy, or
b) heat shock and cytotoxic therapy for the treatment of tumors.

5. A package comprising a matrix metalloproteinase inhibitor (or pharmaceutically acceptable salt or prodrug ester thereof) together with instructions for use in combination with
a) radiotherapy, or
b) heat shock and cytotoxic therapy in the treatment of tumor.

6. (canceled)

7. A method according to claim 1, in which the matrix metalloproteinase inhibitor is one of the compounds disclosed in published international patent applications Nos. WO 98/14424, WO 97/22587 and EP 606046, or a pharmaceutically acceptable prodrug derivative thereof, or a pharmaceutically acceptable salt thereof.

8. A method according to claim 1, in which the matrix metalloproteinase inhibitor is N-hydroxy-2(R)-[[4-methoxyphenylsulfonyl](3-picolyl)amino]-3-methyl-butaneamide hydrochloride)monohydrate, or a pharmaceutically acceptable prodrug derivative thereof, or a pharmaceutically acceptable salt thereof.

9. A method according to claim 1 in which the matrix metalloproteinase inhibitor, or a pharmacologically acceptable salt or prodrug ester, is in the form of a enteral composition.

10. A method of treating cancer in a subject in need of such treatment which comprises radiotherapy in combination with heat shock therapy, and further comprises administering to the subject an effective amount of a matrix metalloproteinase.
